(19) United States
(12) Patent Application Publication
(10) Pub. No.: US 2009/0011014 A1
(43) Pub. Date: Jan. 8, 2009

(54) Tablet Formulation for Sustained Drug-Release

(75) Inventors: Louis Cartilier, Beaconsfield (CA); Mihaela Ungur, La Plaine (CA); Chafic Chebli, Pointe-Claire (CA)

Correspondence Address: DICKINSON WRIGHT PLLC, 1875 Eye Street, NW, Suite 1200, WASHINGTON, DC, 20006, US

(73) Assignee: UNIVERSITE DE MONTREAL, Montreal (CA)

(21) Appl. No.: 11/793,994

(22) PCT Filed: Dec. 20, 2005
(86) PCT No.: PCT/CA2005/001934

§ 371 Date: May 12, 2008

(30) Foreign Application Priority Data
Dec. 24, 2004 (CA) 2,491,665

(51) Int. Cl. A61K 9/26; A61K 47/36; A61K 31/137

(52) U.S. Cl. 424/465; 424/469; 514/778; 514/646

(57) ABSTRACT

Disclosed is a pharmaceutical sustained release tablet for oral administration of a drug which is made of a compressed blend of at least three dry powders including a powder of a drug, a powder of a sustained release matrix for the drug, and a powder of at least one electrolyte. The sustained release matrix consisting of an un-cross-linked high amylose starch wherein the high amylose is substituted by at least one organic substituent comprising at least one carboxyl group. This organic substituent is preferably a carboxyalkyl having 2 to 4 carbon atoms, its salt or mixture thereof. This tablet has the advantage of having an improved integrity.

### FIELD OF THE INVENTION

The present invention relates to a sustained-release drug formulation. More specifically, the invention relates to a pharmaceutical formulation maintaining the integrity of a hydrophilic tablet comprising substituted amylose as a matrix for sustained release of the drug contained in the tablet.

### BRIEF DESCRIPTION OF THE PRIOR ART

### Controlled Drug-Release Systems and Their Characteristics

For many years, increased attention has been given to drug administration characteristics, which has led to the development of new pharmaceutical dosage forms allowing the control of drug release.

Among the many oral dosage forms that can be used for controlled drug-release, tablets are of major interest in the pharmaceutical industry because of their highly efficient manufacturing technology.

Matrix tablets obtained by direct compression of a mixture of a drug and a polymer would be the simplest way to orally achieve controlled release of the active ingredient. Of course, these tablets should also show good mechanical qualities (i.e. tablet hardness and resistance to friability) to meet manufacturing process, subsequent handling and packaging requirements.

Furthermore, matrix polymers should be easily obtained, biocompatible and non-toxic, with the proviso that biodegradable synthetic polymers have the disadvantage of possible toxicity after absorption of the degraded products.

### Polysaccharide Matrices

Several types of polymers have been proposed so far as matrices for the controlled release of drugs. Examples of such polymers-used in hydrophilic matrices are some cellulose derivates like hydroxypropylmethylcellulose, non-cellulosic polysaccharides like guar gum or alginate derivates, acrylic acid polymers like Carbopol® [Buri P. and Doelker E., Pharm. Acta Helv., 55, 189-197 (1980)]. Poly(vinylpyrrolidone) has also been proposed in addition to the above-mentioned polymers [Lapidus H. and Lordi N. G., J. Pharm. Sci., 57, 1292-1301 (1968)]. Other polymers, for example ethylcellulose or polyvinylchloride, have been deployed in inert matrices [Salomon J.-L. and Doelker E., Pharm. Acta Helv., 55, 174-182 (1980)].

Polysaccharide biodegradable matrices for tablets are of interest because the degradation of a natural product like starch occurs naturally in the human body [Kost J. et al., Biomaterials, 11, 695-698 (1990)].

Starch is composed of two distinct fractions: (1) amylose, a non-ramified fraction containing about 4,000 glucose units, and (2) amylopectin, a branched fraction containing about 100,000 glucose units [Biliaderis C., Can. J. Physiol. Pharmacol., 69, 60-78 (1991)].

Unmodified, modified, derivatized and cross-linked starches have been proposed as binders, disintegrants or fillers in tablets [Short et al., U.S. Pat. Nos. 3,622,677 and 4,072,535; Trubiano, U.S. Pat. No. 4,369,308; McKee, U.S. Pat. No. 3,034,911], but no controlled release properties have been described. Thus, these patents are not relevant when considering the present invention.

Some works have disclosed the use of physically- and/or chemically-modified starches for sustained drug-release. The authors of these papers have presented the usual types of starches, i.e. those containing low amounts of amylose, and have not even mentioned the role of amylose, nor amylose itself [Nakano M. et al., Chem. Pharm. Bull., 35, 4346-4350 (1987); Van Aerde P. et al., Int. J. Pharm., 45, 145-152 (1988)]. Some works have even attributed a negative role to amylose present in thermally-modified starches used in sustained drug-release tablets [Hermann J. et al., Int. J. Pharm., 56, 51-63 & 65-70 (1989) and Int. J. Pharm., 63, 201-205 (1990)].

Physical modifications of amylose for pharmaceutical formulations have also been disclosed: non-granular amylose as a binder-disintegrant [Nichols et al., U.S. Pat. No. 3,490,742], and glassy amylose as a coating for oral, delayed-release composition due to enzymatic degradation of the coating into the colon [Alwood et al., U.S. Pat. No. 5,108,758]. These patents are not related to substituted amylose as a matrix excipient for sustained drug-release and, accordingly, are not related to the present invention.

Wai-Chiu C. et al. [Wai-Chiu et al., U.S. Pat. No. 5,468,286] disclosed a starch binder and/or filler useful in manufacturing tablets, pellets, capsules or granules. The tablet excipient is prepared by enzymatically debranching starch with alpha-1,6-D-glucanohydrolase to yield at least 20% by weight of “short chain amylose”. No controlled release properties are claimed for this excipient. Moreover, starch (unmodified, modified or cross-linked) must be enzymatically treated with alpha-1,6-D-glucanohydrolase to be debranched and to yield so-called “short chain amylose”. Thus, starch with a high content of amylopectin is obviously preferred, and amylose is rejected as being unsuitable because debranching is impossible since it has no branching. The role of amylose is not only ignored but also considered negatively.

In connection with this reference, it must also be emphasized that “short-chain amylose” does not exist. In the present specification and appended claims, when the term “amylose” is used, it refers only to amylose having a long chain consisting of more than 250 glucose units (between 1,000 and 5,000 units according to most of the scientific literature), joined by alpha-1,4-D glucose links, in a linear sequence. This is totally different from short chains of 20 to 25 glucose units. Consequently, this work is not related to the present invention, which regards a particular pharmaceutical formulation to maintain the integrity of a substituted amylose matrix tablet.

Several patents relate to the use of cross-linked amylose in tablets for controlled drug-release or as a binder-disintegrant in certain cases [Mateescu et al., U.S. Pat. No. 5,456,921; Mateescu et al., U.S. Pat. No. 5,603,956; Cartilier et al., U.S. Pat. No. 5,616,343; Dumoulin et al., U.S. Pat. No. 5,807,575; Chouinard et al., U.S. Pat. No. 5,885,615; Cremer et al., U.S. Pat. No. 6,238,698].

Lenaerts V. et al. [U.S. Pat. No. 6,284,273] disclosed cross-linked high amylose starch rendered resistant to amylase. Such amylase-resistant starches are obtained by co-cross-linking high amylose starch with polyols. Suitable agents that could be used as additives to high amylose starch for controlled release prior to cross-linking of the high amylose starch include, but are not limited to, polyvinyl alcohol, beta-(1-3) xylan, xanthan gum, locust bean gum and guar gum.

Lenaerts V. et al. [U.S. Pat. No. 6,419,957] disclosed cross-linked high amylose starch having functional groups as a matrix for the slow release of pharmaceutical agents. This matrix tablet excipient is prepared by a process comprising the steps of: (a) reacting high amylose starch with a cross-linking agent cross-linked at a concentration of about 0.1 g to about 40 g of cross-linking agent per 100 g of high amylose starch to afford cross-linked amylose; and (b) reacting the cross-linked high amylose starch with a functional group-attaching reagent at a concentration of about 75 g to about 250 g of functional group-attaching reagent per 100 g of cross-linked amylose to afford cross-linked amylose having a functional group.

Lenaerts V. et al. [U.S. Pat. No. 6,607,748] disclosed cross-linked high amylose starch for use in controlled-release pharmaceutical formulations and manufacturing processes. Such cross-linked high amylose starch is prepared by (a) cross-linking and chemical modification of high amylose starch, (b) gelatinization, and (c) drying to obtain a powder of said controlled-release excipient.

All these patents disclose only cross-linked amylose and some of its variants, which are to be distinguished from linearly-substituted amylose used in the present invention.

As already mentioned above, carboxymethyl starch has been disclosed as a tablet disintegrant [McKee, U.S. Pat. No. 3,034,911]. This is explainable as all starches used or disclosed in this patent contain low levels of amylose, and one knows today that high amylose content is an essential feature to obtain sustained drug-release properties [Cartilier et al., U.S. Pat. No. 5,879,707, Substituted amylose as a matrix for sustained drug release].

For example, Mehta A. et al. [U.S. Pat. No. 4,904,476] disclosed also the use of sodium starch glycolate as a disintegrant. This patent considers only carboxymethyl starch having a low content in amylose as opposed to the present invention which considers high amylose starch, but also discloses a disintegrant, which is the opposite of a sustained-release system.

Staniforth J. et al. [U.S. Pat. No. 5,004,614] disclosed a controlled-release device with a coating that is substantially impermeable to the entrance of an environmental fluid and substantially impermeable to the exit of the active agent during a dispensing period and having an orifice for drug release. Cross-linked or un-cross-linked sodium carboxymethyl starch is proposed among other materials for coating.

The coated controlled-release system described herein is totally different from a matrix tablet when considering the structural aspects and the release mechanisms involved. Also, the necessary presence of an orifice through the coating distinguishes it from the present invention. Furthermore, a hydrophilic matrix system, as described in the present invention, necessarily implies that water penetrates the tablet, contrary to the U.S. Pat. No. 5,004,614 invention, which requires the coating to be impermeable to an aqueous environment. Finally, there is no mention of the necessity of having high amylose content, an essential feature of the present invention.

Cartilier L. et al. [U.S. Pat. No. 5,879,707] disclosed a pharmaceutical sustained-release tablet for oral administration, consisting of a compressed blend of at least two dry powders including a powder of at least one pharmaceutical drug and a powder of a sustained-release matrix for the drug. The sustained-release matrix consists essentially of non-crystalline, un-cross-linked, substituted amylose prepared by reacting, in a basic medium, amylose with at least one organic substituent that reacts functionally with the hydroxyl groups of the amylose molecule.

### SUMMARY OF THE INVENTION

Substituted amylose is known to be an interesting excipient for the preparation by direct compression of drug sustained release hydrophilic matrix tablets.

It has now been discovered that high amylose starch substituted with organic groups comprising at least one carboxyl group can be advantageously combined to electrolytes in order to maintain the swollen hydrophilic matrix tablet integrity when it is immersed in a medium undergoing pH changes simulating the pH evolution of the environment surrounding an oral pharmaceutical dosage form transiting along the gastrointestinal tract.

In the absence of an electrolyte, such a swollen tablet produces cracks and/or partial or complete separation of the tablet parts in order to relieve the tablet internal stress, which forbids their normal and safe therapeutic use. Surprisingly, the addition of an electrolyte provides a complete stabilization of the swollen matrix structure or at least significantly delays the apparition of the abovementioned problems and/or decreases their intensity.

More particularly, it has been found that high amylose carboxymethyl starch matrix tablets can be advantageously improved by the addition of electrolytes. Such an addition permits to maintain the integrity of the swollen matrix tablets while allowing a controlled and sustained release of the drug with a remarkable close-to-linear release profile.

A first object of the present invention is thus to provide a pharmaceutical sustained release tablet with an improved integrity for oral administration of at least one drug, wherein the tablet consists of a compressed blend of at least three dry powders including
a powder of said at least one drug,
a powder of a sustained release matrix for the drug, the sustained release matrix consisting of an un-cross-linked high amylose starch wherein said high amylose is substituted by at least one organic substituent comprising at least one carboxyl group, and
a powder of at least one electrolyte.

Preferably, the high amylose starch is substituted by at least one organic substituent which is a carboxyalkyl containing 2 to 4 carbon atoms, a salt of this carboxyalkyl or a mixture thereof. More preferably, the organic substituent is carboxymethyl, sodium carboxymethyl or mixture thereof.

Advantageously, the degree of substitution of the substituted amylose starch, which is expressed as the ratio of numbers of moles of the at least one organic substituent per kg of the high amylose starch, is equal to or higher than 0.1. More advantageously, the degree of substitution ranges from 0.1 to 0.4.

The electrolytes used in accordance with the present invention may be found in a dry powder form or in a liquid form adsorbed on a dry powder.

Preferably, the electrolytes consist of low molecular weight electrolytes which may be selected from strong or weak acids, strong or weak bases and salts that are strong or weak electrolytes. They also may consist of a buffer.

Preferably also, the electrolytes are selected from weak organic bases and weak organic acids. More preferably, they consist of salts.

Strong electrolytes are preferred to weak electrolytes.

The most preferred electrolytes that may be used in accordance with the invention are sodium chloride, potassium chloride, calcium chloride, calcium lactate, sodium sulfate, citric acid, arginine hydrochloride, urea, sodium acid phosphate and disodium phosphate.

The drug present in the tablet may have a solubility ranging from very soluble to very slightly soluble. It can be in any pharmaceutically suitable form like a salt, a free base or a free acid. The tablet of the present invention may also include more than one drug.

The tablet according to the invention can also include at least one other excipient liker those commonly used in the pharmaceutical area. By way of examples, the excipient may consist of hydroxypropylmethylcellulose (HPMC), lubricants such as magnesium stearate, colorants, anti-oxydants and/or fillers.

Surprisingly, it has also been discovered that high amylose carboxymethyl starch matrix tablets containing very soluble ionic drugs show excellent performance regarding release rates and matrix integrity, particularly when they contain high concentrations of that drug. In such cases, the drug also acts as the electrolyte.

It is thus another object of the present invention to provide a pharmaceutical sustained release tablet with an improved integrity for oral administration of at least one drug, consisting of a compressed blend of at least two dry powders including
a powder of said at least one drug, the drug being a very soluble ionic drug representing at least 20% by weight of the total weight of the tablet, and
a powder of a sustained release matrix for the drug, the sustained release matrix consisting of an un-cross-linked high amylose starch wherein said high amylose is substituted by at least one organic substituent selected from the group consisting of carboxymethyl, sodium carboxymethyl and mixture thereof, said substituted amylose starch having a degree of substitution, expressed as the ratio of the number of moles of carboxymethyl substituents per kg of the high amylose starch, that is equal to or higher than 0.1.

Preferably, the degree of substitution of the substituted amylose starch ranges from 0.1 to 0.4.

The tablet according to this other object of the invention can also include at least one other excipient. Suitable excipients are excipients well known in the pharmaceutical area and include, without being limited to, hydroxypropylmethylcellulose (HPMC), lubricants such as magnesium stearate, colorants, anti-oxydants and fillers.

The invention and its advantages will be better understood upon reading the following non-restrictive detailed description and examples, with reference being made to the accompanying drawings.

### DESCRIPTION OF THE DRAWINGS

FIG. 1 represents the different types of cracks being observed for high amylose carboxymethyl starch matrix tablets: a) C1; b) nC1; c) C2.

FIG. 2 represents the different types of bursting being observed for high amylose carboxymethyl starch matrix tablets: a) DC=double cone (note that some C2 cracks also appear in this particular case); b) M*=mushroom type, but on one face only. The second tablet has been dried but nevertheless retains its characteristic structure.

FIG. 3 is a diagram showing the percentage (%) of acetaminophen released in acidic and moderately alkaline media from SA, CA.lab-1.55 matrix tablets in function of time (hours) for 400-mg tablets containing 10% of drug.

FIG. 4 is a diagram showing the percentage (%) of acetaminophen released from SA, CA.lab-1.8, SA, CA. lab-1.55 and SA, G-2.7 matrix tablets in function of time (hours) for 400-mg tablets containing 10% of drug. SA, CA.lab matrix tablets were immersed for 1 hour in acidic medium (pH=1.2), and then in moderately alkaline medium (pH=7.4). The data for SA, G-2.7, extracted from U.S. Pat. No. 5,879,707, were obtained under experimental conditions exactly similar to the SA, CA.lab tablets testing except that a constant pH medium was used (pH=7.4).

FIG. 5 is a diagram showing the percentage (%) of acetaminophen released from SA, CA-0.05 matrix tablets in function of time (hours) for 400-mg tablets containing 10% of drug and different sodium chloride loadings (0, 10 and 15%).

FIG. 6 is a diagram showing the percentage (%) of acetaminophen released from SA, CA-0.05 matrix tablets in function of time (hours) for 400-mg tablets containing 10% of drug and 10% of sodium chloride when they are immersed for 0.5, 1 or 2 hours in an acidic medium.

FIG. 7 is a diagram showing the percentage (%) of pseudoephedrine hydrochloride (PE) released from SA, CA-0.05 matrix tablets in function of time (hours) for 800-mg tablets containing different drug loadings (20, 37.5, 50 and 60%).

FIG. 8 is a diagram showing total drug-release time (hours) in function of the pseudoephedrine hydrochloride percentage (%) in SA, CA-0.05 matrix tablets of different weights (400, 600 and 800 mg).

### DETAILED DESCRIPTION OF THE INVENTION

### Preliminary Considerations

Cartilier L. et al. [U.S. Pat. No. 5,879,707] disclosed a pharmaceutical sustained-release tablet for oral administration, consisting of a compressed blend of at least two dry powders, including a powder of at least one pharmaceutical drug and the powder of a sustained-release matrix for the drug. The sustained-release matrix essentially consists of non-crystalline, un-cross-linked, substituted amylose prepared by reacting, in a basic medium, amylose with at least one organic substituent having a function that reacts with the hydroxyl groups of the amylose molecule. Typical substituted amylose tablets swell in water, but differ from customary swellable hydrophilic matrices by a surprisingly high mechanical strength in the swollen state. As a consequence, it is possible to create tablets that do not show any disintegration, even if mechanical stresses occur, such as, for example, after administration in the gastrointestinal tract. Various aspects of this invention have also been covered in the scientific literature [Chebli C. and Cartilier L., J. Pharm. Belg., 54(2), 51-53 & 54-56 (1999); Chebli C. et al., Pharm. Res., 16(9), 1436-1440 (1999); Chebli C. and Cartilier L., Int. J. Pharm., 193(02), 167-173 (2000); Chebli C. et al., Int. J. Pharm., 222(2), 183-189 (2001), Cartilier L. et al., Proceedings of ISAB²-2005, page 102, 3^{rd} International Symposium on Advanced Biomaterials/Biomechanics, Apr. 3-6, 2005].

Several observations can, however, be made about the invention disclosed in U.S. Pat. No. 5,879,707:
All the examples and experimental results provided in the above-mentioned references present the use of non-ionic substituted amylose polymers although U.S. Pat. No. 5,879,707 does not restrict itself to non-ionic substituents and mentions the possibility of grafting a carboxylic (—COOH) substituent to protect the hydrophilic matrix from enzymatic degradation.
In vitro drug release tests were done in an aqueous medium maintained at a constant pH (pH=7.34). Since the substituted amylose polymers were non-ionic, the gelification properties were not pH-dependent, and, thus, there was no need to conduct release experiments in a pH gradient simulating the pH evolution of the gastrointestinal tract.
Also, preferably, the substituted amylose has a substituent-to-amylose ratio (expressed in mole of substituent per kg of amylose) that is equal to or higher than 0.4. More preferably, such a ratio ranges from 0.4 to 7.0.
Furthermore, when the pharmaceutical drug(s) used in the tablet is (are) very slightly soluble, the powder of such drug(s) may represent up to 80% by weight of the tablet. When, however, the pharmaceutical drug(s) is(are) highly soluble, the powder of such drug(s) should not exceed 40% by weight of the tablet. Furthermore, the results obtained for sodium salicylate, a freely soluble drug (see example 5 and FIG. 16), show a time for release of 95% of the drug of 6.5 hours for a 400-mg tablet containing 10% of drug, demonstrating controlled release but rather poor performance in terms of sustained release.

On the other hand, carboxymethyl starch, i.e. starch containing a low amount of amylose and which has been reacted with chloroacetic acid or sodium chloroacetate, is used as a disintegrant in immediate-release tablets to promote their fast disintegration and subsequent fast dissolution of the active ingredient now dispersed in an aqueous environment [Bolhuis G. K. et al., Drug Develop. Ind. Pharm. 12(4), 621-630 (1986); Bolhuis G. K. et al., Acta Pharm. Tech., 30(1), 242-32 (1984)]. Such a product has been commercialized among others under the trademark Explotab®. It targets the opposite goal of a hydrophilic matrix system, which tries to maintain the integrity of the dosage form in order to release slowly the active ingredient. Note that erodible matrices are just a special case where physical and/or chemical matrix degradation is progressive and controlled to allow controlled release of the active ingredient.

Despite the fact that carboxymethyl starch is used as a disintegrant, it would be interesting to evaluate the sustained-release properties of carboxymethylamylose, more precisely high amylose carboxymethyl starch as low amylose carboxymethyl starch has served patients for decades and has thereby proved its safety. Also, since carboxymethylamylose is an ionic polymer that will be used for oral sustained drug-release, in vitro release tests now need to consider the pH changes occurring in the gastrointestinal tract.

### The Problem

Matrix tablets comprising high amylose carboxymethyl starch and drug have been obtained and tested for their release properties according to U.S. Pat. No. 5,879,707. Their release properties also have been evaluated in a pH gradient simulating the pH evolution of the tablet environment when traveling along the gastrointestinal tract, i.e. from a strongly acidic to a moderately alkaline environment.

Matrix tablets containing high amylose starch substituted through an etherification reaction with chloracetic acid or sodium chloracetate showed good sustained drug-release properties in a moderately alkaline medium, i.e. pH=7.4 aqueous solution. Surprisingly, the tablets selected for their best sustained-release properties and good mechanical resistance to stress, when swollen in the pH=7.4 aqueous solution, presented cracks, separated into two parts loosely attached at their centre or even split into several parts when they were evaluated with a pH gradient. Some tablets containing high amylose carboxymethyl starch with a very low substitution degree presented the same problems whatever the aqueous medium in which they were immersed.

Such poor mechanical behavior of these tablets forbids their normal therapeutic use. Indeed, when the stomach churns, thereby exerting significant physical force on the formulation, there is a strong risk of the tablet breaking apart, which could lead to a burst of drug release, especially when the drug is freely or very soluble. Furthermore, the integrity of the matrix tablet must be strictly maintained in cases such as dry or press-coated tablets, double or multi-layered tablets, and geometry-controlled-release tablets.

### The Solution

The swelling of the various substituted amylose tablets reported in the above-mentioned references can be described as moderate when compared to the usual hydrophilic matrices like hydroxypropylmethylcellulose. This is particularly true for the high amylose carboxymethyl starch tablets of the present invention, which contain a very rigid gel. It is hypothesized that the rigidity of the gel and its tight network hinder water penetration into the tablet, but more importantly strongly decrease diffusion of the dissolved drug out of the matrix tablet. Consequently, the drug accumulates in a dissolved state inside the tablet, thereby increasing the internal osmotic pressure, which in the end produces cracks and/or partial or complete separation of the tablet parts to relieve the tablet internal stress.

The easiest logical approach to solve the above-mentioned problems would be to increase the drug concentration to decrease the polymer concentration, thereby reducing the tightness of the gel network. Indeed, it was hoped that an increase in tablet porosity would resolve the problem of the lack of gel elasticity, but such an approach was unsuccessful.

The next logical approach to solve the problem described above would be to add swelling polymers (ionic or non-ionic) to maintain the integrity of the high amylose carboxymethyl starch tablet, these polymers hopefully combining with high amylose carboxymethyl starch to create a more elastic and less dense network to facilitate drug diffusion, thus relieving the internal stress of the matrix tablet. This approach failed totally as well as did approaches based on adding similar substances (pregelatinized starches with high or low amylose content, soluble starch derivates). Despite the fact that the approach was similar to increasing drug concentration, adding non-ionic soluble filler was also tried but proved to be unsuccessful. Finally, adding insoluble ionic filler failed as well.

When electrolytes are dissolved in water, the solute exists in the form of ions in the solution. Strong electrolytes like NaCl or HCl exist almost completely in the ionic form in moderately concentrated aqueous solutions. Inorganic acids such as HCl, HNO₃, H₂SO₄, inorganic bases such as NaOH and KOH of the alkali metal family, Ba(OH)₂ and Ca(OH)₂ of the alkaline earth group, and most inorganic and organic salts are highly ionized and belong to the class of strong electrolytes. For weak electrolytes like acetic acid, equilibrium exists between the molecules and ions. Most organic acids and bases and some inorganic compounds, such as H₃BO₃ and NH₄OH, belong to the class of weak electrolytes. Even some salts and complex ions are weak electrolytes [Martin A. et al., Physical Pharmacy, 1983a].

The theory of electrolytes has found several applications in the pharmaceutical field. Often, when an organic drug is poorly soluble, one synthesizes a salt thereof to increase the water solubility of the drug. Also, electrolytes are added to adjust the tonicity of injectable solutions to make them isotonic. Osmotic pumps are a well-known type of drug-delivery device where the use of salts allows the generation of a driving force, i.e. osmotic pressure, permitting constant drug release through a hole drilled in the semi-permeable membrane surrounding the core [Martin A. et al., Physical Pharmacy, 1983b]. Electrolytes may be employed as osmotic agents although non-ionic substances may be deployed too: “Osmagents useful as release modifying agents in the present invention include, for example, sodium chloride, calcium chloride, calcium lactate, sodium sulfate, lactose, glucose, sucrose, mannitol, urea, and many other organic and inorganic compounds known in the art” [U.S. Pat. No. 5,004,614]. This is indeed a classical application of osmotic agents promoting or accelerating drug release. All these applications are based on the fact that electrolytes are generally highly soluble and because they generate osmotic pressure resulting from their dissolution in aqueous media.

Adding an electrolyte to the high amylose carboxymethyl starch matrix formulation, typically sodium chloride, is the opposite of what should be done logically. Indeed, sodium chloride should pump more water and faster inside the tablet, thereby increasing internal osmotic pressure, and making the tablet present cracks, separate into two parts loosely attached at their centre or even burst into several parts, and all that more quickly and at a higher level than in the absence of the said electrolyte. Surprisingly, the addition of an electrolyte provides complete stabilization of the swollen matrix structure or at least significantly delays the appearance of the above-mentioned problems and/or decreases their intensity, thereby allowing its use in oral drug delivery. Nevertheless, strong electrolytes are preferred to weak electrolytes like weak organic acids and bases.

Surprisingly, it was observed that high amylose carboxymethyl starch matrix tablets containing very soluble ionic drugs like pseudoephedrine hydrochloride showed excellent performance regarding release rates and matrix integrity, especially when they contained high concentrations of that drug. In cases of very soluble ionic drugs, it might be useful to replace, at least partially, current fillers like lactose by an electrolyte when formulations containing low drug concentrations are requested.

Pharmaceutical sustained-release tablets, according to the invention, are prepared by compressing, as is known per se, a blend of dry powders, including at least a pharmaceutical drug powder, at least a powder of high amylose carboxymethyl starch used as a sustained-release matrix and an electrolyte. If desired, the tablets may also include a small amount of lubricant, and one or more fillers, also in powder form. If desired, a mixture of two or more drugs may be used instead of one. Once the drug and the other ingredients have been blended, generally by conventional means, including, but not limited to, powder blending, dry or wet granulation, the resulting blend is compressed to form a tablet. The method of preparing such tablets is well-known in the art and need not be described further. Pharmaceutical sustained-release tablets according to the invention can also be of the dry-coated type, prepared by direct compression for example. Once again, the methods of preparing dry-coated tablets are well-known and need not be described further.

### EXAMPLES

### Example 1

### Preparation of Matrix Tablets

Considering that the present invention concerns with a pharmaceutical sustained-release tablet for oral administration, consisting essentially of a compressed blend of at least three dry powders, including the powder of at least one pharmaceutical drug, the powder of a sustained-release matrix for the drug and the powder of at least one electrolyte, preparation of the said matrix tablets will be explained below. The drug(s), high amylose-substituted starches, electrolytes and various excipients used in matrix formulations are presented here as is the tablet preparation procedure.

To illustrate the advantages of the present invention, various drugs have been selected as models for the evaluation of swollen matrix tablet integrity or for release profile study. Note that some other drugs were simply discussed in the description of the present invention. For clarity, certain descriptive terms reported in Table 1 will be used. They allude to the ranges of solubility given in the pharmacopeia monographs [“The United States Pharmacopeia XXIII—The National Formulary XVIII”, 1995]. These terms are defined in a table on page 2071, entitled “Description and Solubility”, which gives the corresponding parts of solvent required for one part of solute. The solubility of the various drugs reported in the examples and/or simply discussed herein is described as follows:

TABLE 1
U.S.P. XXIII Solubility Specifications

| | Parts of solvent | |
|---|---|---|
| | required to solubilize | Example of |
| Descriptive terms | 1 part of drug | drugs |
| Very soluble | Less than 1 | Pseudoephedrine |
| | | hydrochloride, Sodium |
| | | salicylate (in boiling |
| | | water) |
| Freely soluble | From 1 to 10 | Sodium salicylate, |
| | | bupropion hydrochloride |
| Soluble | From 10 to | Acetaminophen |
| | 30 | (in boiling water) |
| Sparingly soluble | From 30 to | Acetaminophen (room |
| | 100 | temperature) |
| Slightly soluble | From 100 to 1,000 | Theophylline |
| Very slightly soluble | From 1,000 to 10,000 | |
| Practically insoluble | 10,000 and | |
| or insoluble | over | |

First, substituted amylose (SA) was prepared by reacting high amylose starch (Hylon VII®, The National Starch and Chemical Company, Bridgewater, N.J., U.S.A.) with sodium chloracetate (Aldrich Chemical Company, Saint Louis, Mo., U.S.A.), in a strongly basic medium [see U.S. Pat. No. 5,879,707]. Different degrees of substitution are obtained by simply varying the substituent/high amylose starch ratio. The products prepared according to this example are referred to as SA, CA.lab-n, where “SA” means high amylose substituted starch, “CA” defines the substituent used, herein chloracetate, “.lab” means batches obtained at the laboratory scale, and “n” represents the degree of substitution (DS) expressed as the ratio “mole of substituent/kg of high amylose starch”. It is worth remembering that Hylon VII® contains approximately 70% of amylose chains and 30% of amylopectin. Note that SA, CA. lab-0.00, which will be used in Example 10, is high amylose starch treated in the same way except that no reactant was added to the reacting medium.

Second, high amylose sodium carboxymethyl starch was obtained directly from Roquette Fréres S. A. (Lestrem, France). However, SA, CA pilot batches were dried with alcohol in place of acetone. The DS is expressed in another way than for laboratory scale batches: it is defined as the number of moles of reactant divided by the number of moles of anhydroglucose; the number of moles of anhydroglucose is obtained by dividing the starch dry weight by 162 (162=molecular weight of one unit of anhydroglucose). SA, CA-0.05 (more precisely 0.046) and 0.07 (more precisely 0.067) are used in the present invention.

Some electrolytes were also included in the present invention: sodium chloride, sodium acid phosphate, disodium phosphate, arginine hydrochloride, and citric acid.

Finally, when specific excipients were part of the formulation evaluated, their role was explained in the appropriate example.

Tablets have been prepared by direct compression, i.e. dry mixing of drug, SA, CA.lab-n or SA, CA-n, electrolytes, if any, and excipients, if any, followed by compression. The drug and the other ingredients of the formulation were mixed manually in a mortar. For swollen matrix integrity evaluation, tablets weighing 400 mg each were compressed for 20 seconds at 2.5 ton/cm² pressure on an IR 30-ton press (C-30 Research & Industrial Instruments Company, London, U.K.). The diameter of the tablets was 1.26 cm. For drug release evaluation, tablets weighing 400, 600 or 800 mg each were compressed for 20 seconds at 2.5 ton/cm² pressure on an IR 30-ton press (C-30 Research & Industrial Instruments Company). The diameter of the tablets was 1.26 cm.

### Example 2

### Evaluation of Swollen Tablet Integrity

The applicants have observed that high amylose carboxymethyl starch matrix tablets presented cracks, separated into two parts loosely attached at their centre or even split into several parts when swollen in aqueous solution, particularly when going through a pH gradient. Surprisingly, it was noted that the addition of an electrolyte provides complete stabilization of the swollen matrix structure or at least significantly delays the appearance of the above-mentioned problems and/or decreases their intensity, thereby allowing its application in oral drug delivery. Thus, a standardized method has been designed to describe the modifications occurring during the tablet's immersion in an aqueous solution as well as the moment of their appearance.

Tablets prepared as disclosed hereinabove in Example 1 were placed individually in 900 ml of a hydrochloric acid solution medium (pH=1.2), at 37° C., in U.S.P. XXIII dissolution apparatus No. 2 equipped with a rotating paddle (50 rpm). After remaining in the acidic solution for a period of 1.5 hours, the tablets were transferred to a phosphate buffer solution medium (pH=7.4), at 37° C., in the same U.S.P. XXIII dissolution apparatus No. 2 equipped with a rotating paddle (50 rpm) until the end of the test. All formulations were tested in triplicate.

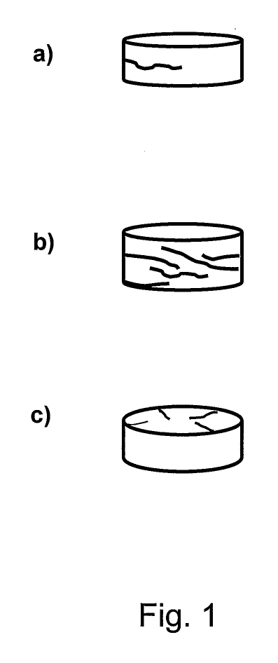

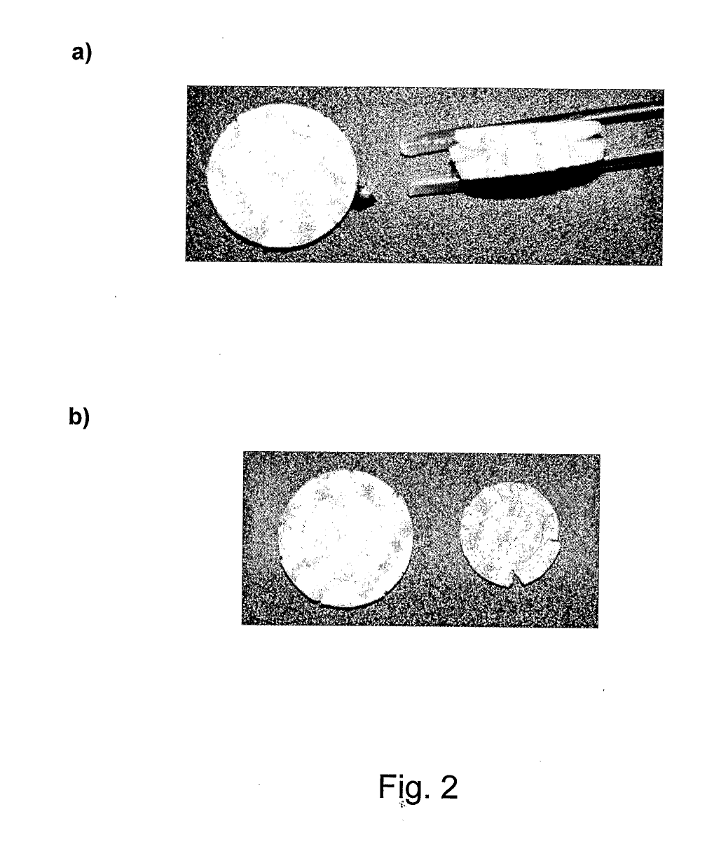

The observation of macroscopic transformations has been standardized with specific qualitative terms describing them and recording the moment of their appearance (hours) in a table. A sequence of two events, cracks followed by bursting, was noted as the appearance of crack(s) in the tablet was often followed by more drastic modification of the matrix structure, the bursting being partial or total. The following terms have been employed: C1=crack type 1; nC1= multiple cracks type 1; C2=cracks type 2; DC=double cone; M=mushroom; M*=mushroom type, but on one face only; flakiness; erosion. C1 represents a single crack appearing along the radial surface of the cylinder. nC1 represents multiple cracks appearing along the radial surface of the tablet. C2 means that one or more cracks appear on one or both facial surfaces of the tablet. DC means that the tablet separates longitudinally into two parts loosely attached at their centre; each part adopts a convex shape due to internal tension of the shrinking gel. M* and M represent partial bursting of the tablet where all the parts remained well attached to the main part of the tablet; the shape looks like a mushroom or like dry earth in some desert areas. These structural modifications have been schematically represented in FIGS. 1 and 2. Some empirical rules may be drawn from the analysis of Examples 4 to 25: C1 leads to DC; C2 leads to M* or M; nC1 leads to flakiness; C1+C2 leads to DC, M, M*, DC+M* or DC+M; the erosion process is not linked to the appearance of cracks. However, the addition of electrolytes smoothes the transformation process as some crack phenomena do not necessarily lead to the bursting appearance; electrolytes may also hinder the appearance of DC, leading to the appearance of a M structure. This allows the consideration of a rather semi-quantitative approach, keeping in mind that the more the tablet fully splits apart, the higher are the risks of undesired burst release in vivo.

### Example 3

### Drug Release Evaluation

The drug release properties of some typical matrix tablets were assessed by an in vitro dissolution test. Acetaminophen was used as drug model with solubility intermediate between soluble and sparingly soluble; its solubility is not influenced by pH in physiological conditions. Pseudoephedrine hydrochloride was used as a very soluble ionic drug model.

Two types of experimental conditions were tested: a) constant pH (pH=7.4), a method exactly similar to that in U.S. Pat. No. 5,879,707; b) pH gradient, where the pH progressed from an acidic value (pH=1.2) to a moderately alkaline one (pH=7.4), simulating roughly physiological conditions.

### a) Constant pH

Tablets prepared as disclosed hereinabove in Example 1 were placed individually in 900 ml of a phosphate buffer solution medium (pH=7.4), at 37° C., in U.S.P. XXIII dissolution apparatus No. 2 equipped with a rotating paddle (50 rpm). The amount of acetaminophen released at predetermined time intervals was followed spectrophotometrically (acetaminophen: 242 nm). All formulations were tested in triplicate. The drug release results are expressed in terms of cumulative % or mg released in function of time (hours).

### b) pH Gradient

Tablets prepared as disclosed hereinabove in Example 1 were placed individually in 900 ml of a hydrochloric acid solution medium (pH=1.2), at 37° C., in U.S.P. XXIII dissolution apparatus No. 2 equipped with a rotating paddle (50 rpm). After remaining in the acidic solution for a period of 0.5, 1 or 2 hours, the tablets were transferred to a phosphate buffer solution medium (pH=7.4), at 37° C., in the same U.S.P. XXIII dissolution apparatus No. 2 equipped with a rotating paddle (50 rpm) until the end of the test. The amount of acetaminophen or pseudoephedrine hydrochloride released at predetermined time intervals was followed spectrophotometrically (acetaminophen: 242 nm and pseudoephedrine hydrochloride: 257 nm). All formulations were tested in triplicate. The drug release results are expressed in terms of cumulative % or mg released in function of time (hours).

### Example 4

### Effect of Tablet Drug Loading on In Vitro Tablet Integrity

Tablets were prepared according to the procedure described in Example 1 and evaluated according to the test conditions described in Example 2. The active ingredient (A.I.) was acetaminophen, a non-ionic drug, sparingly soluble to soluble, depending on the temperature (from room temperature to boiling water), with its solubility uninfluenced by pH at physiological conditions. The formulations and their evaluation are presented in Table 2.

TABLE 2
Evaluation of the integrity of swollen tablets containing increasing
acetaminophen concentrations and SA, CA-0.05 or SA, CA-0.07

| | | SA, | SA, | | | | |
|---|---|---|---|---|---|---|---|
| | A.I. | CA-0.05 | CA-0.07 | Cracks | | Bursting | |
| No. | (%) | (%) | (%) | Time | Type | Time | Type |
| 1 | 10 | 90 | | 4.6 | C1 | 8.0 | DC |
| 2 | 10 | | 90 | 1.5 | C1 | 4.5 | DC |
| 3 | 20 | 80 | | 4.6 | C1 | 8.0 | DC |
| 4 | 20 | | 80 | 1.5 | C1 | 2.0 | DC |
| 5 | 30 | 70 | | 3.0 | C1 | 4.0 | DC |

| | | | |
|---|---|---|---|
| 6 | 50 | 50 | Disintegration |
| 7 | 60 | 40 | Disintegration |
| 8 | 80 | 20 | Impossible to obtain |
| | | | compressed tablets |

Matrix tablets containing acetaminophen and high amylose carboxymethyl starch are not practically useful, whatever their substitution degree, as they quickly show major cracks (type C1) followed by split-up in the worst form (DC). Increased drug loading accelerates the process and/or amplifies it and does not constitute a valid approach to solve the problem.

### Example 5

### Effect of Adding a Non-Ionic Hydrophilic Polymer

Tablets were prepared according to the procedure described in Example 1 and evaluated according to the test conditions described in Example 2. The A.I. was acetaminophen. The formulations and their evaluation are presented in Table 3.

TABLE 3
Evaluation of the integrity of swollen tablets containing
acetaminophen, SA, CA-0.05 and increasing concentrations
of HPMC K4M

| | | | HPMC | | | | |
|---|---|---|---|---|---|---|---|
| | | SA, | Methocel | | | | |
| | A.I. | CA-0.05 | K4M | Cracks | | Bursting | |
| No. | (%) | (%) | (%) | Time | Type | Time | Type |
| 1 | 10 | 0 | 90 | No | | | |
| 2 | 10 | 10 | 80 | No | | | |
| 3 | 10 | 25 | 65 | No | | | |
| 4 | 10 | 45 | 45 | 6 | nC1 | 2.0 | Flakiness |
| 5 | 10 | 50 | 40 | 6 | nC1 | 4.0 | Flakiness |
| 6 | 10 | 60 | 30 | 6 | nC1 | | Flakiness |
| 7 | 10 | 65 | 25 | 2 | nC1 | | Flakiness |
| 8 | 10 | 70 | 20 | 2 | nC1 | | Flakiness |
| 9 | 10 | 72 | 18 | 4.5 | C1 + | | M + DC |
| | | | | | C2 | | |
| 10 | 10 | 75 | 15 | 5 | C1 | | DC |

Matrix tablets containing a high percentage of high amylose carboxymethyl starch SA, CA-0.05 and a low percentage of HPMC K4M still present the same problems, i.e. crack C1 and bursting DC. Increasing the HPMC K4M concentration brings another type of problem as all tablets now demonstrate flakiness. When the percentage of HPMC is higher than that of SA, CA-0.05, the tablets do not present structural problems anymore, but are outside the scope of this invention. Thus, the addition of a non-ionic hydrophilic polymer is not a valuable solution to the matrix integrity problem of high amylose carboxymethyl starch matrix tablets.

### Example 6

### Effect of Adding a Non-Ionic Hydrophilic Polymer (Cont.)

Tablets were prepared according to the procedure described in Example 1 and evaluated according to the test conditions described in Example 2. The A.I. was acetaminophen. The formulations and their evaluation are presented in Table 4.

TABLE 4
Evaluation of the integrity of swollen tablets containing
acetaminophen, SA, CA-0.07 and increasing concentrations
of HPMC K4M

| | | | HPMC | | | | |
|---|---|---|---|---|---|---|---|
| | | SA, | Methocel | | | | |
| | A.I. | CA-0.07 | K4M | Cracks | | Bursting | |
| No. | (%) | (%) | (%) | Time | Type | Time | Type |
| 1 | 10 | 60 | 30 | 4.5 | nC1 | | Flakiness |
| 2 | 10 | 70 | 20 | 3 | C1 + | | M + DC |
| | | | | | C2 | | |

One can make the same observations regarding the addition of HPMC K4M to SA, CA-0.07 as in the case of SA, CA-0.05. A low percentage of HPMC K4M is not able to avoid the problems of cracking and bursting. Increasing the HPMC K4M percentage starts flakiness phenomena. Changing the substitution degree and adding a non-ionic hydrophilic polymer is not a valuable solution to the above-mentioned problems.

### Example 7

### Effect of Adding a Non-Ionic Hydrophilic Polymer (But Less Hydrophilic than HPMC K4M)

Tablets were prepared according to the procedure described in Example 1 and evaluated according to the test conditions described in Example 2. The A.I. was acetaminophen. The formulations and their evaluation are presented in Table 5.

TABLE 5
Evaluation of the integrity of swollen tablets containing
acetaminophen, SA, CA-0.05 and increasing concentrations
of HPMC E4M

| | | | HPMC | | | | |
|---|---|---|---|---|---|---|---|
| | | SA, | Methocel | | | | |
| | A.I. | CA-0.05 | E4M | Cracks | | Bursting | |
| No. | (%) | (%) | (%) | Time | Type | Time | Type |
| 1 | 10 | 0 | 90 | No | | | |
| 2 | 10 | 45 | 45 | 6 | nC1 | | Flakiness |
| 3 | 10 | 50 | 40 | 6 | nC1 | | Flakiness |
| 4 | 10 | 60 | 30 | 6 | nC1 | | Flakiness |

Matrix tablets containing high amylose carboxymethyl starch SA, CA-0.05 and a percentage of HPMC E4M varying from 30 to 45% still present the same problems of flakiness. When the percentage of HPMC E4M is higher than that of SA, CA-0.05, tablets do not present structural problems anymore, but are outside the scope of this invention. Thus, the addition of a non-ionic polymer less hydrophilic than HPMC K4M is not a valuable solution to the matrix integrity problem of high amylose carboxymethyl starch matrix tablets. The small gain in time before the first cracks appear in the tablet may be explained by the fact that HPMC E4M is less hydrophilic.

### Example 8

### Effect of Adding an Ionic Hydrophilic Polymer

Tablets were prepared according to the procedure described in Example 1 and evaluated according to the test conditions described in Example 2. The A.I. was acetaminophen. The formulations and their evaluation are presented in Table 6. Note that in the case of formulations 2 and 3, the tablets were immersed for only 30 minutes in acidic medium.

TABLE 6
Evaluation of the integrity of swollen tablets containing
acetaminophen, SA, CA-0.05 and increasing concentrations
of Carbopol

| | | SA, | Carbopol | Magnesium | | | | |
|---|---|---|---|---|---|---|---|---|
| | A.I. | CA-0.05 | 974 P NF | stearate | Cracks | | Bursting | |
| No. | (%) | (%) | (%) | (%) | Time | Type | Time | Type |
| 1 | 10 | 75 | 15 | | 7 | C1 | | DC |

| | | | | | | |
|---|---|---|---|---|---|---|
| 2 | 10 | 74.5 | 15 | 0.5 | No | Significant |
| | | | | | | erosion |
| 3 | 10 | 79.5 | 10 | 0.5 | No | Significant |
| | | | | | | erosion |

The addition of an ionic hydrophilic polymer to SA, CA-0.05 matrix tablets does not stop the appearance of cracks and bursting phenomena. Furthermore, adding a hydrophobic lubricant like magnesium stearate to such compositions starts a significant erosion process. This adds to the impossibility of using such polymers in the formulation of SA, CA-0.05 matrix tablets to solve the above-mentioned problems.

### Example 9

### Effect of Adding Pregelatinized Starch (with Low Amylose Content)

Tablets were prepared according to the procedure described in Example 1 and evaluated according to the test conditions described in Example 2. The A.I. was acetaminophen. The formulations and their evaluation are presented in Table 7.

TABLE 7
Evaluation of the integrity of swollen tablets containing
acetaminophen, SA, CA-0.05 or SA, CA-0.07 and increasing
concentrations of Lycatab ® PGS

| | | | SA, | | | | | |
|---|---|---|---|---|---|---|---|---|
| | | SA, | CA- | Lycatab ® | | | | |
| | A.I. | CA-0.05 | 0.07 | PGS | Cracks | | Bursting | |
| No. | (%) | (%) | (%) | (%) | Time | Type | Time | Type |
| 1 | 10 | 89.75 | | 0.25 | 4.5 | C1 | 7.0 | DC |
| 2 | 10 | 88.75 | | 1.25 | 4.5 | C1 | 7.0 | DC |
| 3 | 10 | 87.50 | | 2.50 | 4.5 | C1 | 8.0 | DC |
| 4 | 10 | 85.00 | | 5.00 | 5.0 | C1 | 8.0 | DC |
| 5 | 10 | 80.00 | | 10.00 | 5.0 | C1 | 8.0 | DC |
| 6 | 10 | 75.00 | | 15.00 | 4.5 | C1 | 8.0 | DC |
| 7 | 10 | | 89.75 | 0.25 | 1.0 | C1 | 3.5 | DC |
| 8 | 10 | | 88.75 | 1.25 | 1.0 | C1 | 3.5 | DC |
| 9 | 10 | | 87.50 | 2.50 | 1.0 | C1 | 3.5 | DC |
| 10 | 10 | | 85.00 | 5.00 | 1.0 | C1 | 3.5 | DC |
| 11 | 10 | | 80.00 | 10.00 | 1.0 | C1 | 3.5 | DC |

The addition of pregelatinized starch with low amylose content did not help to maintain the integrity of SA, CA-0.05 high amylose carboxymethyl starch matrices. Increasing the degree of substitution of high amylose carboxymethyl starch while adding the said pregelatinized starch accelerated the process as C1 cracks and DC bursting appeared slightly sooner than in the absence of pregelatinized starch (see Example 4).

### Example 10

### Effect of Adding Pregelatinized High Amylose Starch

Tablets were prepared according to the procedure described in Example 1 and evaluated according to the test conditions described in Example 2. The A.I. was acetaminophen. The formulations and their evaluation are presented in Table 8.

TABLE 8
Evaluation of the integrity of swollen tablets containing
acetaminophen, SA, CA-0.05 and increasing
concentrations of SA, CA-0.00

| | | SA, | SA, | | | | |
|---|---|---|---|---|---|---|---|
| | A.I. | CA-0.05 | CA-0.00 | Cracks | | Bursting | |
| No. | (%) | (%) | (%) | Time | Type | Time | Type |
| 1 | 10 | 80 | 10 | 5.5 | C1 + C2 | 8.0 | DC |
| 2 | 10 | 70 | 20 | 6.0 | C1 + C2 | 8.0 | DC |
| 3 | 10 | 60 | 30 | 6.0 | C1 + C2 | 8.0 | DC |
| 4 | 10 | 50 | 40 | 6.0 | C1 + C2 | 8.0 | DC |

The addition of pregelatinized starch with high amylose content did not help to maintain the integrity of SA, CA-0.05 high amylose carboxymethyl starch matrices. The said pregelatinized starch slightly delayed the apparearance of cracks and C2 showed in addition to C1. DC bursting appeared at the same moment as in the absence of pregelatinized starch (see Example 4).

### Example 11

### Effect of Adding Dextrin, an Oligosaccharide Derived from Starch

Tablets were prepared according to the procedure described in Example 1 and evaluated according to the test conditions described in Example 2. The A.I. was acetaminophen. The formulations and their evaluation are presented in Table 9.

TABLE 9
Evaluation of the integrity of swollen tablets containing
acetaminophen, SA, CA-0.05 or SA, CA-0.07 and
increasing concentrations of dextrin

| | | | SA, | Dextrin | | | | |
|---|---|---|---|---|---|---|---|---|
| | | SA, | CA- | Type 2 | | | | |
| | A.I. | CA-0.05 | 0.07 | Sigma | Cracks | | Bursting | |
| No. | (%) | (%) | (%) | (%) | Time | Type | Time | Type |
| 1 | 10 | 87 | | 3 | 4.66 | C1 | 7.66 | DC |
| 2 | 10 | 85 | | 5 | 4.66 | C1 | 7.66 | DC |
| 3 | 10 | 80 | | 10 | 4.66 | C1 | 7.66 | DC |
| 4 | 10 | 75 | | 15 | 4.0 | C1 | 5.66 | DC |
| 5 | 10 | 70 | | 20 | 3.75 | C1 | 5.66 | DC |
| 6 | 10 | | 87 | 3 | 1.5 | C1 | 4.5 | DC |
| 7 | 10 | | 85 | 5 | 1.5 | C1 | 4.0 | DC |
| 8 | 10 | | 80 | 10 | 1.5 | C1 | 4.0 | DC |
| 9 | 10 | | 75 | 15 | 1.0 | C1 | 2.0 | DC |
| 10 | 10 | | 70 | 20 | 1.0 | C1 | 2.0 | DC |

Increasing concentrations of an oligosaccharide like dextrin did not modify the nature of the problems associated with the use of SA, CA-0.05 or 0.07 matrix tablets, but they progressively decreased the time before their appearance. Thus, adding similar polymers of lower molecular weight does not constitute a valid solution to resolve the above-mentioned problems.

### Example 12

### Effect of Adding a Non-Ionic Soluble Filler, Sugar

A non-ionic soluble filler, sugar, was added to the formulation of SA, CA-0.05 matrix tablets containing acetaminophen. Tablets were prepared according to Example 1 and evaluated as described in Example 2. However, this strategy proved to be ineffective too.

For example, a tablet containing 10% of acetaminophen, 80% of SA, CA-0.05 and 10% of saccharose showed cracks (C1+C2 type) after 2 hours only and split apart (DC+M type) after 4 hours only. The same kind of observations as in the case of dextrin can be made. Increasing the solubility of a non-ionic saccharidic excipient (saccharose>dextrin>starch) has a negative effect on the integrity of the matrix tablet (see also Example 4 for comparison).

### Example 13

### Effect of Adding an Insoluble Ionic Filler

Considering that increasing the solubility of the non-ionic excipient added to the formulation negatively affected the integrity of SA, CA matrix tablets, some trials were performed with an insoluble ionic filler, Emcompress® (a certain type of calcium phosphate). Again, it did not resolve the problems, but even increased them. For example, a tablet containing 10% of acetaminophen, 75% of SA, CA-0.05 and 15% of Emcompress® showed a C1 type crack after only 1.5 hours, leading quickly to DC bursting.

### Example 14

### Effect of Adding a Low Molecular Weight Organic Acid

Tablets were prepared according to the procedure described in Example 1 and evaluated according to the test conditions described in Example 2. The A.I. was acetaminophen. The formulations and their evaluation are presented in Table 10.

TABLE 10
Evaluation of the integrity of swollen tablets containing
acetaminophen, SA, CA-0.05 and increasing
concentrations of citric acid

| | | SA, | Citric | | | | |
|---|---|---|---|---|---|---|---|
| | A.I. | CA-0.05 | acid | Cracks | | Bursting | |
| No. | (%) | (%) | (%) | Time | Type | Time | Type |
| 1 | 10 | 85 | 5 | 5.0 | C2 | 7.0 | DC + M* |
| 2 | 10 | 80 | 10 | 6.0 | C2 | 8.0 | DC + M* |

The addition of a low molecular weight organic acid like citric acid, a weak electrolyte, helped to partially resolve the problems, i.e. cracks and bursting. Indeed, the nature of cracks changed from C1 to C2, and the moment of their appearance was delayed slightly.

### Example 15

### Effect of Adding a Salt Formed from a Low Molecular Weight Organic Base and an Acid

Tablets were prepared according to the procedure described in Example 1 and evaluated according to the test conditions described in Example 2. The A.I. was acetaminophen. The formulations and their evaluation are presented in Table 11.

TABLE 11
Evaluation of the integrity of swollen tablets containing
acetaminophen, SA, CA-0.05 and increasing
concentrations of arginine hydrochloride

| | | SA, | Arginine | | | | |
|---|---|---|---|---|---|---|---|
| | A.I. | CA-0.05 | hydrochloride | Cracks | | Bursting | |
| No. | (%) | (%) | (%) | Time | Type | Time | Type |
| 1 | 10 | 80 | 10 | 5.0 | C1 | 12.0 | DC |
| 2 | 10 | 75 | 15 | 4.5 | C2 | | M |
| 3 | 10 | 70 | 20 | 5.0 | C2 | | M* |
| 4 | 20 | 70 | 10 | 4.5 | C2 | | M |
| 5 | 30 | 60 | 10 | 4.5 | C1 | | DC |

The addition of arginine hydrochloride helped to partially resolve the above-mentioned problems. Indeed, not only the nature of the cracks changed from C1 to C2 when arginine hydrochloride concentration was increased, but also the nature of the bursting changed from DC to M or M*, thus preserving the general shape of the tablet. However, too large an increase in drug concentration while retaining the same arginine hydrochloride concentration made the problems reappear. Thus, the concentration of the electrolyte added to the formulation needs to be adapted to the concentration and solubility of the A.I. present. Note that tablet No. 1 showed significant swelling (300%), but demonstrated shrinking after 5 hours of testing.

### Example 16

### Effect of Adding a Phosphate Buffer (pH=7.4)

Tablets were prepared according to the procedure described in Example 1 and evaluated according to the test conditions described in Example 2. The A.I. was acetaminophen. The formulations and their evaluation are presented in Table 12.

TABLE 12
Evaluation of the integrity of swollen tablets containing
acetaminophen, SA, CA-0.05 or SA, CA-0.07 and increasing
concentrations of phosphate buffer (pH = 7.4)

| | | | | NaH₂PO₄•H₂O/ | | | | |
|---|---|---|---|---|---|---|---|---|
| | | SA, | SA, | Na₂HPO₄ | | | | |
| | A.I. | CA-0.05 | CA-0.07 | (pH = 7.4) | Cracks | | Bursting | |
| No. | (mg) | (mg) | (mg) | (mg/mg) | Time | Type | Time | Type |
| 1 | 40 | 359.0 | | 0.2/0.8 | 5 | C1 + C2 | 7.0 | DC |
| 2 | 40 | 355.2 | | 1.1/3.8 | 5 | C1 + C2 | 7.0 | M |
| 3 | 40 | 350.3 | | 2.1/7.6 | 5 | C1 + C2 | 6.0 | M |
| 4 | 40 | 340.6 | | 4.2/15.2 | 4.5 | C1 + C2 | 6.0 | M |
| 5 | 40 | 321.2 | | 8.4/30.4 | 3.5 | C1 | 5.0 | M |
| 6 | 40 | | 359.0 | 0.2/0.8 | 1.5 | C1 | 3.0 | DC |
| 7 | 40 | | 355.2 | 1.1/3.8 | 1.5 | C1 | 4.0 | DC |
| 8 | 40 | | 350.3 | 2.1/7.6 | 1.5 | C1 | 4.0 | DC |
| 9 | 40 | | 340.6 | 4.2/15.2 | 1.5 | C1 | 5.0 | DC |
| 10 | 40 | | 321.2 | 8.4/30.4 | 1.0 | C1 | 2.0 | DC |

Regarding SA, CA-0.05 matrix tablets, the addition of a buffer (pH=7.4) did not prolong the time before cracks or bursting appeared, but 1% of such a buffer was sufficient to favorably change the nature of the cracks, especially the bursting, which was now partial (=M). Such an improvement was not noted for SA, CA-0.07. A higher DS means that more carboxylic functions will be grafted on the polymer, thus changing the behavior of the matrix in the presence of a buffer. The addition of a buffer may positively affect the integrity of swollen high amylose carboxymethyl starch matrices, provided the nature and concentration of the buffer (pH value) are carefully selected in function of DS of the polymer and the nature and concentration of the drug included in the tablet.

### Example 17

### Effect of Adding a Phosphate Buffer (pH=6.0)

Tablets were prepared according to the procedure described in Example 1 and evaluated according to the test conditions described in Example 2. The A.I. was acetaminophen. The formulations and their evaluation are presented in Table 13.

TABLE 13
Evaluation of the integrity of swollen tablets containing
acetaminophen, SA, CA-0.05 or SA, CA-0.07 and increasing
concentrations of phosphate buffer (pH = 6.0)

| | | | | NaH₂PO₄•H₂O/ | | | | |
|---|---|---|---|---|---|---|---|---|
| | | SA, | SA, | Na₂HPO₄ | | | | |
| | A.I. | CA-0.05 | CA-0.07 | (pH = 6.0) | Cracks | | Bursting | |
| No. | (mg) | (mg) | (mg) | (mg/mg) | Time | Type | Time | Type |
| 1 | 40 | 358.8 | | 1.0/0.1 | 5 | C1 | 6.0 | DC |
| 2 | 40 | 354.2 | | 5.2/0.6 | 5 | C1 | 6.0 | DC |
| 3 | 40 | 348.4 | | 10.4/1.2 | 5 | C1 | 6.0 | DC |
| 4 | 40 | 336.8 | | 20.8/2.4 | 4.75 | C1 | 6.0 | DC |
| 5 | 40 | 313.6 | | 41.6/4.8 | 4.5 | C1 + C2 | 6.0 | DC |
| 6 | 40 | | 358.8 | 1.0/0.1 | 1 | C1 | 3.5 | DC |
| 7 | 40 | | 354.2 | 5.2/0.6 | 1 | C1 | 3.5 | DC |
| 8 | 40 | | 348.4 | 10.4/1.2 | 1 | C1 | 3.5 | DC |
| 9 | 40 | | 336.8 | 20.8/2.4 | 1 | C1 | 3.5 | DC |
| 10 | 40 | | 313.6 | 41.6/4.8 | 1 | C1 | 3.5 | DC |

For SA, CA-0.05 matrices, the addition of a phosphate buffer (pH=6.0) very slightly increased the time before cracks appeared. Regarding SA, CA-0.07, no improvement was noticed for the range of concentrations investigated. The remarks in Example 16 about the choice of the buffer nature and concentration as well as the effect of drug nature and concentration apply equally here.

### Example 18

### Effect of Adding a Phosphate Buffer (pH=5.4)

Tablets were prepared according to the procedure described in Example 1 and evaluated according to the test conditions described in Example 2. The A.I. was acetaminophen. The formulations and their evaluation are presented in Table 14.

TABLE 14
Evaluation of the integrity of swollen tablets containing
acetaminophen, SA, CA-0.05 or SA, CA-0.07 and increasing
concentrations of phosphate buffer (pH = 5.4)

| | | | | NaH₂PO₄•H₂O/ | | | | |
|---|---|---|---|---|---|---|---|---|
| | | SA, | SA, | Na₂HPO₄ | | | | |
| | A.I. | CA-0.05 | CA-0.07 | (pH = 5.4) | Cracks | | Bursting | |
| No. | (mg) | (mg) | (mg) | (mg/mg) | Time | Type | Time | Type |
| 1 | 40 | 358.8 | | 1.2/0.1 | 4.0 | C1 | 5.5 | DC |
| 2 | 40 | 353.8 | | 6.0/0.2 | 4.5 | C1 + C2 | 7.0 | DC |
| 3 | 40 | 347.7 | | 11.9/0.5 | 4.5 | C1 + C2 | 8.0 | M |
| 4 | 40 | 335.3 | | 23.8/0.9 | 5.5 | C2 | 9.0 | M |
| 5 | 40 | 310.6 | | 47.6/1.8 | 5.5 | C2 | 9.0 | M |
| 6 | 40 | | 358.8 | 1.2/0.1 | 1 | C2 | 3.5 | DC |
| 7 | 40 | | 353.8 | 6.0/0.2 | 1 | C2 | 3.5 | DC |
| 8 | 40 | | 347.7 | 11.9/0.5 | 1 | C2 | 3.5 | DC |
| 9 | 40 | | 335.3 | 23.8/0.9 | 1 | C2 | 3.5 | DC |
| 10 | 40 | | 310.6 | 47.6/1.8 | 1 | C2 | 3.5 | DC |

The addition of increasing concentrations of phosphate buffer (pH=5.4) to SA, CA-0.05 matrix tablets shows an improvement in the swollen tablet's integrity, in the nature of the cracks and bursting as well as in the time of their appearance. Regarding SA, CA-0.07 matrix tablets, a minor improvement was only noticed in the type of crack appearing. The remarks in Example 16 about the choice of buffer nature and concentration as well as the effect of drug nature and concentration apply equally here.

### Example 19

### Effect of Adding Salt

Tablets were prepared according to the procedure described in Example 1 and evaluated according to the test conditions described in Example 2. The A.I. was acetaminophen. The formulations and their evaluation are presented in Table 15.

TABLE 15
Evaluation of the integrity of swollen tablets containing
acetaminophen, SA, CA-0.05 or SA, CA-0.07 and increasing
concentrations of sodium chloride

| | | SA, | SA, | | | | | |
|---|---|---|---|---|---|---|---|---|
| | A.I. | CA-0.05 | CA-0.07 | NaCl | Cracks | | Bursting | |
| No. | (%) | (%) | (%) | (%) | Time | Type | Time | Type |
| 1 | 10 | 89.75 | | 0.25 | 4.66 | C1 | 7.0 | DC |
| 2 | 10 | 88.75 | | 1.25 | 4.66 | C1 | 7.0 | DC |
| 3 | 10 | 87.50 | | 2.50 | 4.66 | C1 | 7.0 | DC |
| 4 | 10 | 85.00 | | 5.00 | 4.66 | C1 | 7.0 | DC |
| 5 | 10 | 80.00 | | 10.00 | 5.5 | C1 | 8.0 | M* |
| 6 | 10 | 75.00 | | 15.00 | 5.0 | C1 | 8.0 | M* |
| 7 | 10 | | 89.75 | 0.25 | 1.5 | C1 | 4.0 | DC |
| 8 | 10 | | 88.75 | 1.25 | 1.5 | C1 | 4.5 | DC |
| 9 | 10 | | 87.50 | 2.50 | 1.5 | C1 | 4.5 | DC |
| 10 | 10 | | 85.00 | 5.00 | 4.66 | C1 | 7.0 | DC |
| 11 | 10 | | 80.00 | 10.00 | 5.5 | C1 | | M* |
| 12 | 10 | | 75.00 | 15.00 | 9.5 | C1 | | M* |

Adding sodium chloride in concentrations above 5% to SA, CA-0.05 matrix tablets improved the swollen tablets integrity in the nature of the bursting (M*) and slightly increased the time before C1 cracks appeared. This trend was also observed for SA, CA-0.07 matrix tablets, but with a dramatic increase of C1 appearance time (almost 7×). Again, there was a correlation between the electrolyte nature and concentration on the one hand and the degree of substitution of the polymer matrix on the other hand when trying to stabilize a high amylose carboxymethyl starch matrix.

### Example 20

### Effect of Adding Salt (Cont.)

Tablets were prepared according to the procedure described in Example 1 and evaluated according to the test conditions described in Example 2. The A.I. was acetaminophen. The formulations and their evaluation are presented in Table 16.

TABLE 16
Evaluation of the integrity of swollen tablets containing SA,
CA-0.05 and increasing concentrations of
acetaminophen and sodium chloride

| | A.I. | SA, CA-0.05 | NaCl | Cracks | | Bursting | |
|---|---|---|---|---|---|---|---|
| No. | (%) | (%) | (%) | Time | Type | Time | Type |
| 1 | 10 | 80 | 10 | 5.5 | C1 | 8.0 | M* |
| 2 | 20 | 70 | 10 | 4.0 | C1 | 12.0 | DC |
| 3 | 30 | 60 | 10 | 4.5 | C1 | | M* |

| | | | | |
|---|---|---|---|---|
| 4 | 40 | 50 | 10 | No |
| 5 | 50 | 40 | 10 | No |
| 6 | 30 | 55 | 15 | No |
| 7 | 40 | 45 | 15 | No |
| 8 | 20 | 60 | 20 | No |

Increasing both drug and sodium chloride concentrations fully stabilized the swollen matrix tablets. No cracks or bursting appeared, demonstrating the surprising benefit of the addition of electrolytes.

### Example 21

### Effect of Adding Salt (Cont.)

Tablets were prepared according to the procedure described in Example 1 and evaluated according to the test conditions described in Example 2. The A.I. was acetaminophen. The formulations and their evaluation are presented in Table 17.

TABLE 17
Evaluation of the integrity of swollen tablets containing
acetaminophen, SA, CA-0.05 and sodium chloride,
compressed at different compression forces

| | | SA, | | | | | | |
|---|---|---|---|---|---|---|---|---|
| | | CA- | Compression | | | | | |
| | A.I. | 0.05 | force | NaCl | Cracks | | Bursting | |
| No. | (%) | (%) | (Tons) | (%) | Time | Type | Time | Type |

| | | | | | |
|---|---|---|---|---|---|
| 1 | 40 | 45 | 1.0 | 15 | No |
| 2 | 40 | 45 | 1.5 | 15 | No |
| 3 | 40 | 45 | 2.0 | 15 | No |

When drug and sodium chloride concentrations were carefully selected, compression force, as usually implemented to manufacture substituted amylose matrix tablets, did not influence the integrity of the swollen matrix tablets. This again demonstrates the usefulness of the invention.

### Example 22

### Effect of Adding Salt (Cont.)

Tablets were prepared according to the procedure described in Example 1 and evaluated according to the test conditions described in Example 2. The A.I. was acetaminophen. The formulations and their evaluation are presented in Table 18.

TABLE 18
Evaluation of the integrity of swollen tablets containing
acetaminophen, SA, CA-0.05, sodium chloride and
a hydrophilic non-ionic polymer, HPMC K4M

| | | SA, | | | | | | |
|---|---|---|---|---|---|---|---|---|
| | | CA- | HPMC | | | | | |
| | A.I. | 0.05 | K4M | NaCl | Cracks | | Bursting | |
| No. | (%) | (%) | (%) | (%) | Time | Type | Time | Type |
| 1 | 10 | 70 | 10 | 10 | 6 | C1 + C2 | 24 | M* |
| 2 | 10 | 45 | 15 | 10 | 6 | C1 + C2 | 24 | M* |
| 3 | 10 | 45 | 10 | 15 | 6 | C1 + C2 | 24 | M* |

Other excipients like non-ionic hydrophilic polymers can be combined with high amylose carboxymethyl starch when sodium chloride is used. The benefit of sodium chloride addition remains as regards the improvement of matrix integrity. When the SA, CA-0.05/HPMC K4M ratio is considered, one notices that the flakiness phenomena have disappeared.

### Example 23

### Effect of Adding Salt (Cont.)

Tablets were prepared according to the procedure described in Example 1 and evaluated according to the test conditions described in Example 2. The A.I. was acetaminophen. The formulations and their evaluation are presented in Table 19.

TABLE 19
Evaluation of the integrity of swollen tablets containing
acetaminophen, SA, CA-0.05, sodium chloride and a
pregelatinized starch with low amylose content

| | | SA, | Lycatab ® | | | | | |
|---|---|---|---|---|---|---|---|---|
| | A.I. | CA-0.05 | PGS | NaCl | Cracks | | Bursting | |
| No. | (%) | (%) | (%) | (%) | Time | Type | Time | Type |
| 1 | 10 | 70 | 10 | 10 | 4.5 | C1 | | M* |
| 2 | 10 | 75 | 7.5 | 7.5 | 8 | C1 | | M* |
| 3 | 10 | 75 | 5 | 10 | 5.5 | C1 | | M* |

In the same manner, excipients like pregelatinized starch can be combined with high amylose carboxymethyl starch when sodium chloride is used. The benefit of sodium chloride addition remains as regards the improvement of matrix integrity.

### Example 24

### Effect of Adding Electrolytes

### Application to Other Active Ingredients

To illustrate the versatility and advantages of the present invention, theophylline was selected as another drug model for tablet integrity evaluation. Tablets were prepared according to the procedure described in Example 1 and evaluated according to the test conditions described in Example 2. The formulations and their evaluation are presented in Table 20.

TABLE 20
Evaluation of the integrity of swollen tablets containing
theophylline, SA, CA-0.05, and sodium chloride alone or
a mixture of sodium chloride and arginine hydrochloride

| | | | Arginine | | | | | |
|---|---|---|---|---|---|---|---|---|
| | Theophylline | SA, CA-0.05 | hydrochloride | Nacl | Cracks | | Bursting | |
| No. | (%) | (%) | (%) | (%) | Time | Type | Time | Type |
| 1 | 10 | 90 | | | 5.5 | C2* | | M* |
| 2 | 20 | 80 | | | 4.5 | C1 | | DC |
| 3 | 10 | 80 | | 10 | 7 | C2* | No | |
| 4 | 20 | 70 | | 10 | 4.5 | C1 | No | |
| 5 | 30 | 60 | | 10 | 1.5 | C1 | 2 | DC |

| | | | | | |
|---|---|---|---|---|---|
| 6 | 10 | 70 | 10 | 10 | No |

Theophylline, a slightly soluble drug, appears to show the same problems as acetaminophen with regard to matrix integrity. A moderate increase in drug loading accelerates and amplifies the problems (see tablet No. 2). The addition of sodium chloride decreases the above-mentioned problems, but one notices that an increase in drug loading needs to be accompanied by an increase in electrolyte loading (see tablet No. 3 to 6) to maintain the benefit of electrolyte addition. Tablet No. 6 shows also the benefit of using a mixture of electrolytes, i.e. sodium chloride and arginine hydrochloride, to maintain the integrity of high amylose carboxymethyl starch matrix tablets.

### Example 25

### Effect of Adding Electrolytes

### Application to Other Active Ingredients (Cont.)

To illustrate the versatility and advantages of the present invention, bupropion hydrochloride was selected as another drug model for tablet integrity evaluation. Tablets were prepared according to the procedure described in Example 1 and evaluated according to the test conditions described in Example 2. The formulations and their evaluation are presented in Table 21.

TABLE 21
Evaluation of the intearity of swollen tablets containing bupropion,
SA, CA-0.05, sodium chloride and arginine hydrochloride

| | Bupropion | SA, | | | | | | |
|---|---|---|---|---|---|---|---|---|
| | hydrochloride | CA-0.05 | Arginine•HCl | NaCl | Cracks | | Bursting | |
| No. | (%) | (%) | (%) | (%) | Time | Type | Time | Type |
| 1 | 10 | 90 | | | 5 | C2 | | M* |
| 2 | 20 | 80 | | | 4 | C1 | | DC |
| 3 | 30 | 70 | | | 4 | C1 | | DC |
| 4 | 40 | 60 | | | 2 | C1 | 2.5 | DC |

| | | | | | |
|---|---|---|---|---|---|
| 5 | 50 | 50 | | | Disintegration after 2 h |
| 6 | 25 | 50 | 16.67 | 8.33 | No |

Bupropion hydrochloride, a freely soluble drug, appears to show the same problems as acetaminophen and theophylline as regards matrix integrity. Like these two drugs, an increase in bupropion hydrochloride loading accelerates and amplifies the problems (see tablet No. 2-5). The addition of a mixture of sodium chloride and arginine hydrochloride resolves the above-mentioned problems and shows the benefit of using a mixture of electrolytes to maintain the integrity of high amylose carboxymethyl starch matrix tablets.

### Example 26

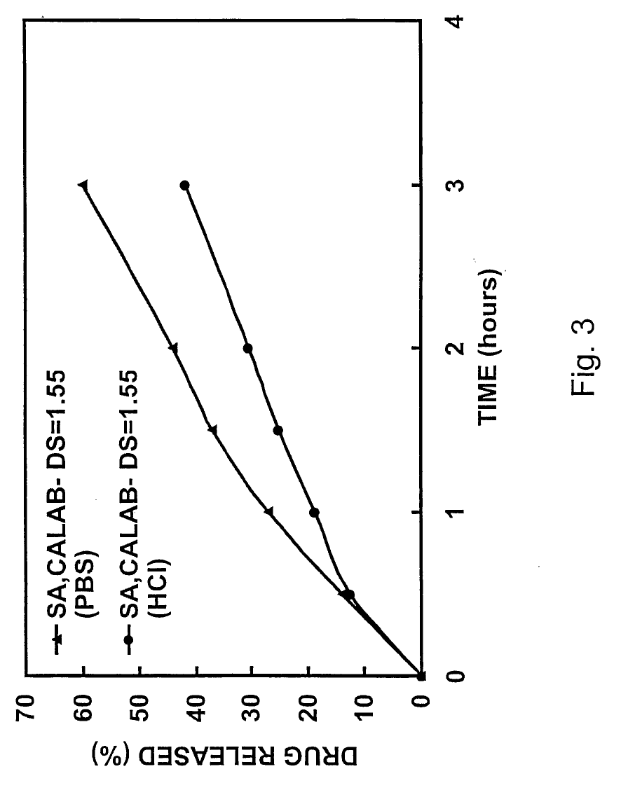

SA, CA.lab tablets were prepared according to the procedure described in Example 1 and evaluated according to the test conditions described in Example 3 except that pH was maintained constant at 1.2 or 7.4. The A.I. was acetaminophen. FIG. 3 shows the percentage (%) of acetaminophen released in acidic and moderately alkaline conditions from SA, CA.lab-1.55 matrix tablets in function of time (hours) for 400-mg tablets containing 10% of drug. The burst release rate was similar for both experiments, i.e. in vitro release in acidic and moderately alkaline environments. This part of the release profile is essentially due to dissolution of the drug present at the matrix surface and directly exposed to the aqueous environment. The solubility of acetaminophen being the same in acidic and moderately alkaline environments, the results are not surprising. The second part of the release profile shows a significant difference where the release rate in acidic medium is much slower than in the moderately alkaline medium. It can be concluded that the high amylose carboxymethylstarch gel structured differently depending on pH.

### Example 27

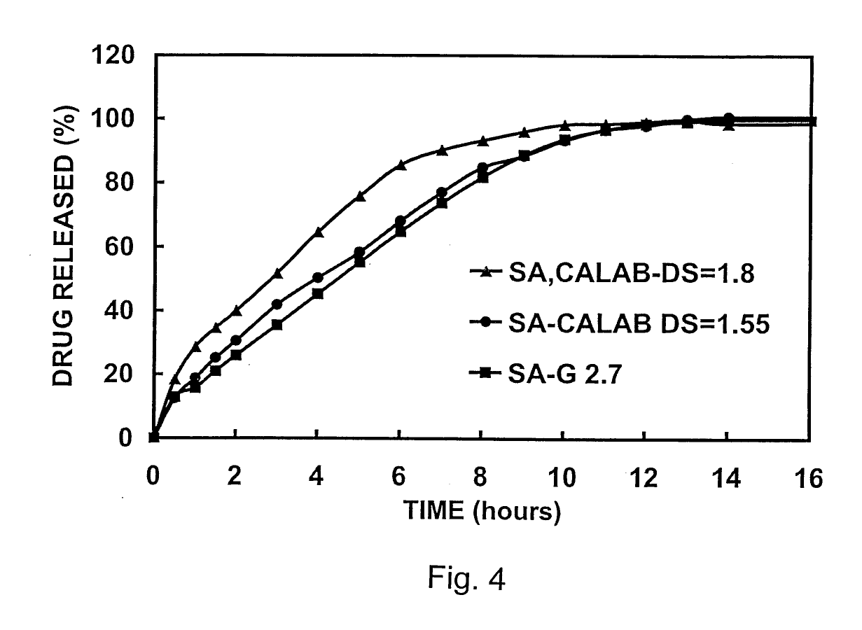

SA, CA.lab tablets were prepared according to the procedure described in Example 1 and evaluated according to the test conditions described in Example 3 (pH gradient, 1 hour in pH=1.2). The A.I. was acetaminophen. FIG. 4 shows the percentage (%) of acetaminophen released from SA, CA.lab-1.8, SA, CA.lab-1.55 and SA, G-2.7 matrix tablets in function of time (hours) for 400-mg tablets containing 10% of drug. SA, CA. lab matrix tablets were immersed for 1 hour in acidic medium (pH=1.2), and then transferred to a moderately alkaline medium (pH=7.4). The data for SA, G-2.7, extracted from U.S. Pat. No. 5,879,707, were obtained for experimental conditions exactly similar to SA, CA tablet testing except that a constant pH medium was used (pH=7.4).

On the one hand, SA, CA.lab-1.55 tablets released the drug more slowly than SA, CA.lab-1.8 matrices; on the other hand, SA, CA.lab-1.55 tablets showed some significant cracking and bursting when going through a pH gradient. This defect makes them useless when considering in vivo applications although they demonstrated an in vitro performance equivalent to SA, G-2.7 tablets as regards the release of a soluble drug like acetaminophen.

### Example 28

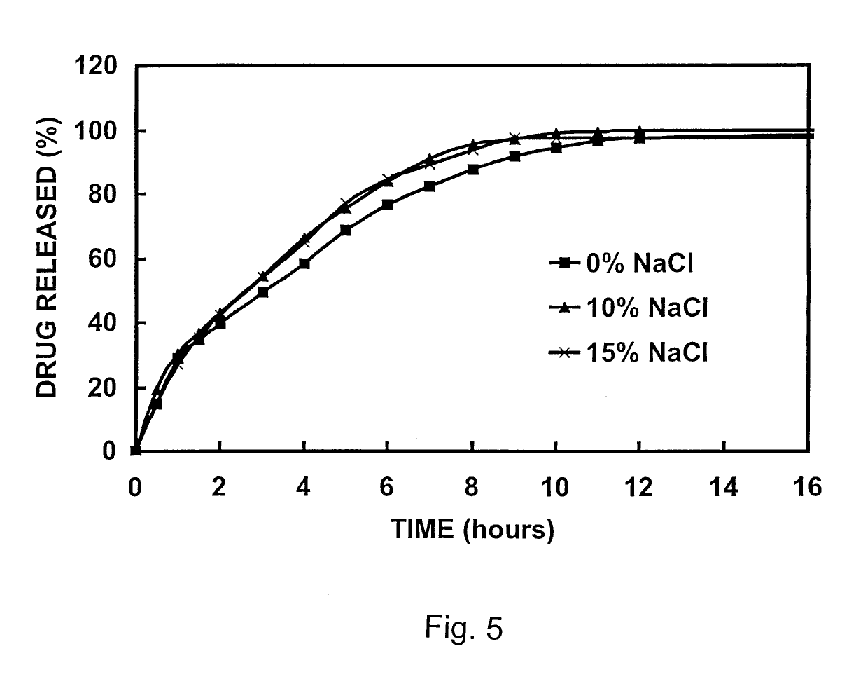

Tablets were prepared according to the procedure described in Example 1 and evaluated according to the test conditions described in Example 3 (pH gradient, 1 hour in pH=1.2). The A.I. was acetaminophen. FIG. 5 shows the percentage (%) of acetaminophen released from SA, CA-0.05 matrix tablets in function of time (hours) for 400-mg tablets containing 10% of drug and different sodium chloride loadings (0, 10 and 15%).

Surprisingly, a typical sustained-release profile is still observed despite significant sodium chloride concentrations (10-15%) in tablets already containing 10% of a soluble drug like acetaminophen. Also, no differences in release rates could be reported between compositions containing 10 and 15% of sodium chloride. The presence of sodium chloride does not even increase the burst release of acetaminophen. Note that for tablets containing only acetaminophen, a slight acceleration of drug release could be observed after 4 hours of evaluation. Indeed, a major crack appeared around that time and promoted dissolution of the drug present at the surface generated by the crack. One must, however, consider that agitation in the dissolution apparatus is quite moderate compared to the stomach, which can churn during the digestion process. In such conditions, the tablet would be broken apart, leading to more dramatic consequences. Note also that, due to sodium chloride, only moderate, partial bursting (M*) appeared after 8-9 hours of testing which was without consequences on the release profile.

### Example 29

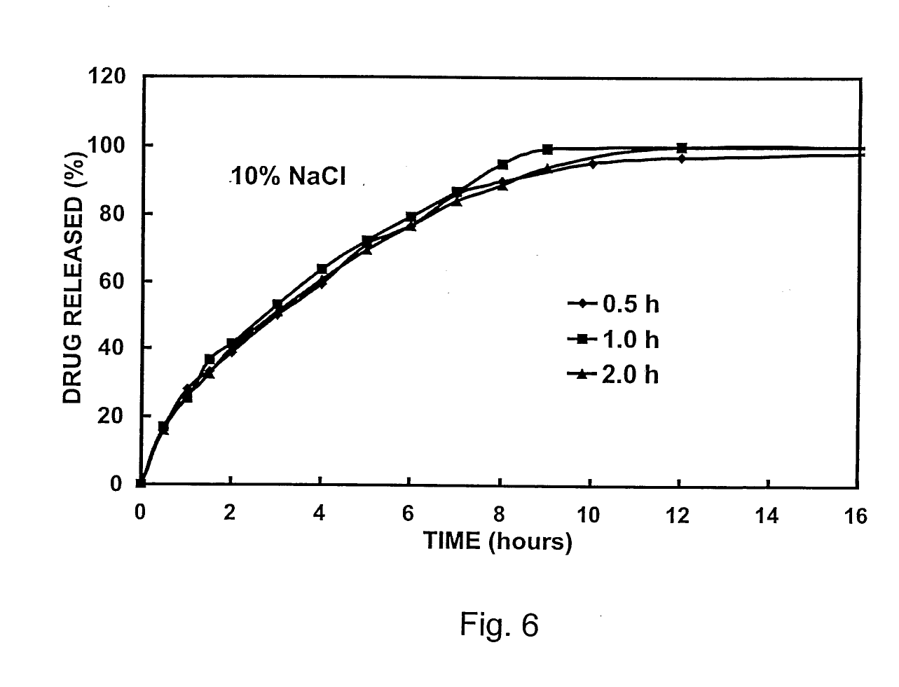

Tablets were prepared according to the procedure described in Example 1 and evaluated according to the test conditions described in Example 3 (pH gradient, 0.5, 1 or 2 hours in pH=1.2). The A.I. was acetaminophen. FIG. 6 shows the percentage (%) of acetaminophen released from SA, CA-0.05 matrix tablets in function of time (hours) for 400-mg tablets containing 10% of drug and 10% of sodium chloride when the tablets are immersed for 0.5, 1 or 2 hours in the acidic medium.

It is noteworthy to note that the release profile from such matrices is not influenced by acidic residence time although high amylose sodium carboxymethylstarch is the sodium salt of an ionic polymer. Furthermore, no modification of the release profile was observed when the tablet was transferred from an acidic to an alkaline medium.

### Example 30

Tablets were prepared according to the procedure described in Example 1 and evaluated according to the test conditions described in Example 3 (pH gradient, 0.5, 1 or 2 hours in pH=1.2). The A.I. was acetaminophen. All tablets contained 10% of drug and 10% of sodium chloride. One batch of tablets contained 0.2% of magnesium stearate and their release profile was compared to that of tablets without magnesium stearate. Magnesium stearate, a well-known tablet lubricant, did not influence the release rate of acetaminophen from SA, CA-0.05 matrix tablets.

### Example 31

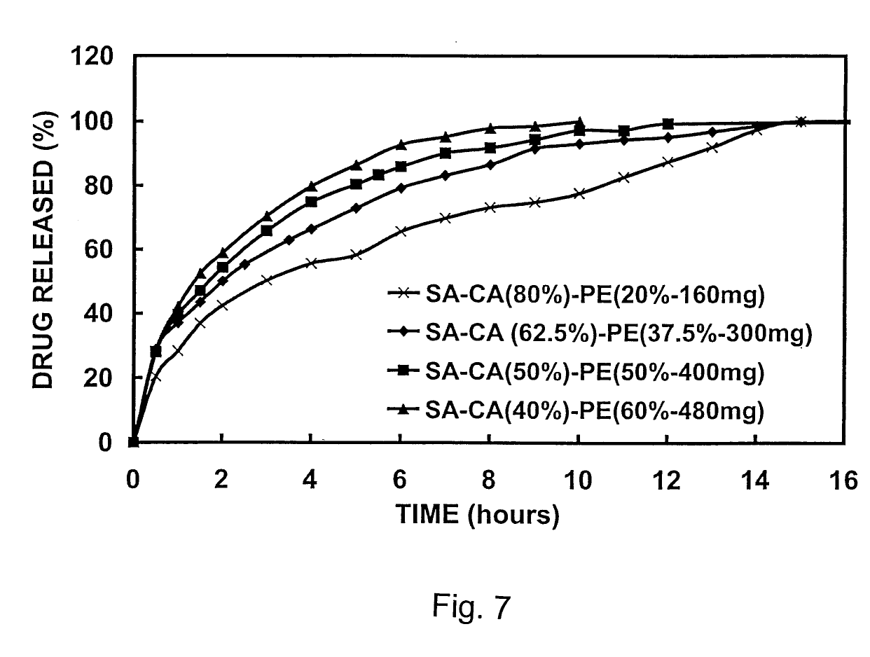

Tablets were prepared according to the procedure described in Example 1 and evaluated according to the test conditions described in Example 3 (pH gradient, 1 hour in pH=1.2). The A.I. was pseudoephedrine hydrochloride (PE). FIG. 7 shows the percentage (%) of PE released from SA, CA-0.05 matrix tablets in function of time (hours) for 800-mg tablets containing different drug loadings (20, 37.5, 50 and 60%). High amylose carboxymethylstarch matrices containing a very soluble ionic drug like pseudoephedrine chloride presented a surprisingly excellent sustained-release performance when they were evaluated, regarding release rates and matrix integrity, in a pH gradient simulating the pH evolution of the tablet environment when traveling along the gastrointestinal tract, i.e. from a strongly acidic to a moderately alkaline environment. Note that the release profile corresponding to tablets containing 20% of drug displays a few release rate accelerations corresponding to small cracks appearing in the matrix. PE is very soluble, and slight secondary burst effects thus appear due to drug dissolution starting on the surface newly exposed to an aqueous environment.

### Example 32

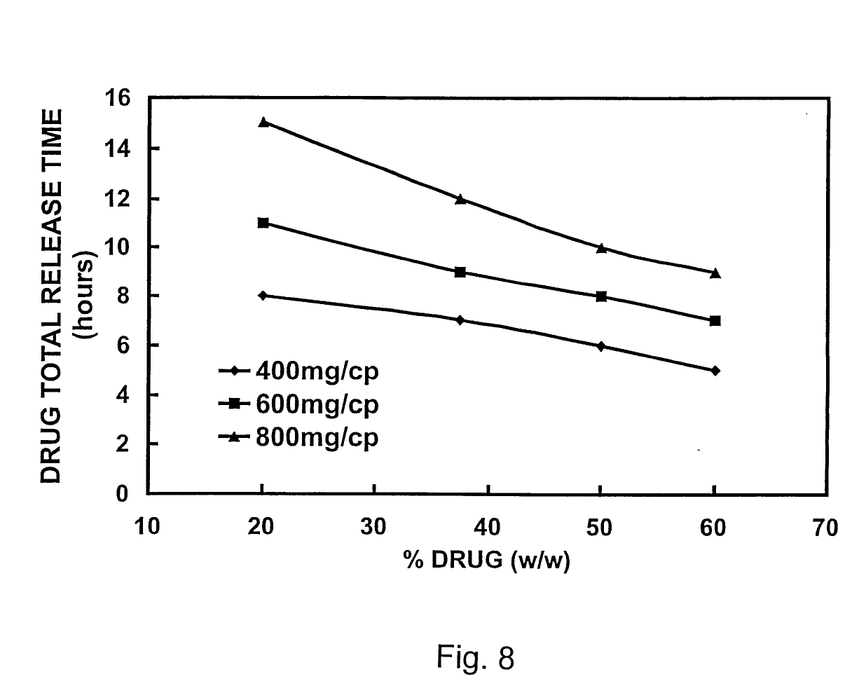

Tablets were prepared according to the procedure described in Example 1 and evaluated according to the test conditions described in Example 3 (pH gradient, 1 hour in pH=1.2). The A.I. was PE. FIG. 8 shows the drug total release time (hours) in function of the PE percentage (%) in SA, CA-0.05 matrix tablets of different weights (400, 600 and 800 mg). PE is a non-ionic, very soluble drug and that makes it usually quite difficult to formulate, especially at high loadings. FIG. 8 illustrates that high drug loading SA, CA-0.05 matrices with excellent performance are easily achieved. Note that, surprisingly, drug loadings lower that 20% were not very successful as cracks appeared in the matrices. This is, however, in good agreement with what has been observed when adding electrolytes like sodium chloride to stabilize the matrix. Consequently, when formulating low drug dosage forms, one has to add electrolytes, in place of current fillers like lactose, to SA, CA-0.05 tablets to obtain a useful tablet weight and stable matrix. On the other hand, this makes it clear that in accordance with the invention, one may incorporate very large amounts of a very soluble ionic drug in a tablet and still achieve very good release control.

Of course, numerous modifications could be made to the above invention, as disclosed and exemplified, without departing from the scope of the appended claims.

1. A pharmaceutical sustained release tablet with an improved integrity for oral administration of at least one drug, wherein said tablet consists of a compressed blend of at least three dry powders including:
a powder of said at least one drug,
a powder of a sustained release matrix for the drug, said sustained release matrix consisting of an un-cross-linked high-amylose starch wherein said high amylose starch is substituted by at least one organic substituent comprising at lease one carboxyl group, and
a powder of at least one electrolyte.

2. The tablet of claim 1, wherein said at least one organic substitutent is a carboxyalkyl containing 2 to 4 carbon atoms, a salt of said carboxyalkyl or a mixture thereof.

3. The tablet of claim 1, wherein said at least one organic substituent is selected from the group consisting of carboxymethyl, sodium carboxymethyl and mixture thereof.

4. The tablet of claim 1, wherein said substituted amylose starch has a degree of substitution, expressed as a ratio of the number of moles of the at least one organic substituent per kg of the high starch, that is equal to or higher than 0.1.

5. The tablet of claim 4, wherein said substituted amylose has a degree of substitution, expressed as the ratio of the number of moles of the at least one organic substituent per kg of the high amylose starch, that ranges from 0.1 to 0.4.

6. The tablet of claim 1, wherein said at least one electrolyte is in a dry powder form or in a liquid form adsorbed on a dry powder.

7. The tablet of claim 1, wherein said at least one electrolyte is a low molecular weight electrolyte selected from the group consisting of strong or weak acids, strong or weak bases and salts.

8. The tablet of claim 7, wherein said at least one electrolyte is a low molecular weight electrolyte selected from the group consisting of weak organic bases and weak organic acids.

9. The tablet of claim 7, wherein said at least one electrolyte is a salt.

10. The tablet of claim 7, wherein said at least one electrolyte is a buffer.

11. The tablet of claim 7, wherein said at least one electrolyte is selected from the group consisting of sodium chloride, potassium chloride, calcium chloride, calcium lactate, sodium sulfate, citric acid, arginine hydrochloride, urea, sodium acid phosphate and disodium phosphate.

12. The tablet of claim 1, wherein said blend of dry powders also includes at least one other excipient.

13. The tablet of claim 12, wherein said at least one other excipient is selected from the group consisting of lubricants, colorants, anti-oxydants and fillers.

14. A pharmaceutical sustained release tablet with an improved integrity for oral administration of at least one drug, wherein said tablet consists of a release matrix consisting of an un-cross-linked high starch wherein said high is substituted by at least one organic substituted selected from the group consisting of carboxymethyl, sodium carboxymethyl and mixture thereof, said substituted amylose starch having a degree of substitution, expressed as the ratio of the number of moles of carboxymethyl substituents per kg of the high amylose starch, that is equal to or higher than 0.1.

15. The tablet of claim 14, wherein said substituted amylose has a degree of substitution, expressed as the ratio of the number of moles of carboxymethyl substituents per kg of the high starch, that ranges from 0.1 to 0.4.

16. The tablet of claim 14, wherein said blend of dry powders also includes at least one other excipient.

17. The tablet of claim 16, wherein said a least one other excipient is selected from the group consisting of lubricants, colorants, anti-oxidants and fillers.

18. The tablet of claim 1, wherein the at least one drug is a water-soluble, ionic drug.

19. The tablet of claim 18, wherein the water-soluble, ionic drug is pseudoephidrine hydrochloride.

20. The tablet of claim 14, wherein the at least one drug is a water-soluble, ionic drug.

21. The tablet of claim 20, wherein the water-soluble, ionic drug is pseudoephidrine hydrochloride.
